(19) United States
(12) Patent Application Publication
(10) Pub. No.: US 2025/0340501 A1
(43) Pub. Date: Nov. 6, 2025

(54) NON-THERMAL PLASMA CATALYTIC CONVERSION OF BIOGAS TO ACETIC ACID AND METHANOL

(71) Applicant: The Chinese University of Hong Kong, Hong Kong (CN)

(72) Inventors: Chunshan SONG, Hong Kong (CN); Pengxian YE, Hong Kong (CN); Debjyoti RAY, Bhabla (IN)

(21) Appl. No.: 19/196,216

(22) Filed: May 1, 2025

Related U.S. Application Data
(60) Provisional application No. 63/641,591, filed on May 2, 2024.

(51) Int. Cl. C07C 29/48; B01J 35/64; B01J 37/34; C07C 51/16

(52) U.S. Cl. CPC C07C 29/48; B01J 35/64; B01J 37/349; C07C 51/16; C07C 2529/40

(57) ABSTRACT

An integrated plasma catalysis system for biogas conversion includes a plasma-treated mesoporous or microporous catalyst and a plasma source for converting a biogas containing methane and carbon dioxide to a liquid oxygenates rich in methanol or acetic acid. Biogas from different solid wastes can be converted under ambient conditions to liquid oxygenates including methanol and acetic acid.

### CROSS-REFERENCE TO RELATED APPLICATION

This application claims the benefit of U.S. Provisional Application Ser. No. 63/641,591, filed May 2, 2024, which is hereby incorporated by reference in its entirety including any tables, figures, or drawings.

### BACKGROUND OF THE INVENTION

Biogas, which consists mainly of CO₂ and CH₄, is a widely available renewable source with large amounts produced worldwide from anaerobic digesters in wastewater treatment plants, dairy manure and food waste in farms, and landfill gas. Biogas conversion is not only a method to utilize various solid wastes, but an important approach to mitigating climate change issues by simultaneously converting two greenhouse gases. Currently, most biogas conversion technologies are directed to combustion for generation of heat or thermo-chemical conversion of biogas to syngas (CO+H₂), for thermo-chemical synthesis under high temperature and pressure. Efficient conversion of biogas to specific desired products is challenging, particularly under mild conditions. Hence, a simultaneous conversion of highly stable CH₄ and CO₂ to liquid oxygenates could yield a cost-effective route to convert biogas directly to target products without costly CO₂ removal and several energy-intensive synthesis steps.

Non-thermal plasmas activate inert gases at near room temperature by the highly energetic electrons that can promote reaction of the relatively stable components of biogas. Among various plasma reactors, dielectric barrier discharge (DBD) is widely used because of flexibility, ease of operation, and scalability. DBD plasma has been shown to effectively activate biogas. Though effective, the plasma reaction still lacks selectivity. Currently, most plasma technologies for biogas conversion can only produce syngas.

Direct conversion of biogas to liquid oxygenates rather than syngas is limited. Some metal-based catalysts have been investigated in plasma conversion of CO₂ and CH₄. Transition metals like Cu promote methanol production, while Fe and Co lead to the formation of acetic acid, but noble metals such as Au or Pt favor the generation of formaldehyde. However, factors influencing the selectivity for liquid products remain unclear. The role of catalyst in tuning product selectivity in plasma conversion of biogas remains a question. How these complex plasma systems influence product selectivity is not clear. The role of metal-based catalyst, plasma discharge and process parameters during plasma catalysis is not well defined.

Hence, a method to convert biogas to high-value chemicals or fuels with high selectivity in a single step under ambient conditions, would constitute a much more energy-efficient and cost-effective route. Biogas conversion to liquid products, in a manner that permits tailoring the liquid product between methanol and acetic acid using an integrated plasma catalysis system, has the potential to meet the demands for these products.

### BRIEF SUMMARY OF THE INVENTION

An embodiment is directed to an integrated plasma catalysis system for biogas conversion that includes a plasma-treated mesoporous or microporous catalyst and a well-defined plasma system for conversion of biogas to liquid oxygenates, such as methanol and acetic acid, where the composition of the liquid oxygenates can be tuned by the process conditions and the nature of catalyst. The catalyst support can be impregnated with a metal and then treated by hydrogen plasma. The metal can be copper, cobalt, and/or nickel. The metal species loaded on the catalyst can be in a flower-like morphology. The type of plasma reactor is dielectric barrier discharge (DBD) plasma.

Another embodiment is to a method for preparing the plasma-treated metal-based catalyst, where a catalyst support (gamma alumina, ZSM-5 zeolite, or SBA-15 molecular sieve) is optionally impregnated with a metal source of copper, cobalt, and/or nickel, and then treated with a hydrogen plasma. The hydrogen plasma is using a hydrogen flow at about 0 to about 100 mL/min. The discharge power of hydrogen plasma can be from about 10 to about 80 W. A discharge gap of between about 1 to about 4 mm can be used with a discharge length of between about 1 to about 10 cm. The catalyst can be treated for a period of about 1 to about 4 hours.

Another embodiment is directed to a method of converting biogas to liquid oxygenates comprising methanol and acetic acid using the integrated plasma catalysis system, where biogas with methane and carbon dioxide is introduced to the plasma-treated metal-based gamma alumina catalyst under plasma to tailor the liquid oxygenates including methanol and acetic acid. The plasma-treated catalyst can be plasma treated γ-Al₂O₃, plasma treated Co/γ-Al₂O₃, Ni/γ-Al₂O₃, and/or Cu/γ-Al₂O₃. When the plasma-treated gamma alumina supported catalyst is a plasma treated γ-Al₂O₃, plasma treated Co/γ-Al₂O₃, and/or Ni/γ-Al₂O₃, with Co or Ni at about 1 to about 20 wt % loading, the dominant liquid oxygenate is acetic acid rather than methanol. When the plasma-treated gamma alumina supported catalyst is plasma treated Cu/γ-Al₂O₃, with Cu at about 0.1 to about 25 wt % loading, the dominant liquid oxygenate becomes methanol rather than acetic acid. The plasma reactor is a dielectric barrier discharge (DBD) plasma. The plasma can form with a discharge gap of about 1 to about 4 mm and a discharge length of about 1 to about 10 cm. The discharge power can be about 5 to about 65 W for methanol rich liquid oxygenates or about 15 to about 55 W for acetic acid rich liquid oxygenates using the appropriate catalyst. The frequency of the dielectric barrier discharge (DBD) reactor can be from about 7 kHz to about 10 kHz. When the biogas has a CH₄:CO₂ ratio of about 0.2 to about 3, a methanol rich liquid oxygenates is generated on Cu/γ-Al₂O₃, and when a CH₄:CO₂ ratio is about 0.1 to about 5, an acetic acid rich liquid oxygenates is generated on other γ-Al₂O₃ catalysts. The gas hourly space velocity (GHSV) can be about 100 to about 2700 h⁻¹. A temperature of about 0 to about 290° C. can be used for a methanol rich liquid oxygenates and a temperature of about 10 to about 300° C. can be used for an acetic acid rich liquid oxygenates. Water vapor of about 1 vol % to 15 vol % can be included with the biogas to enhance methanol production.

Another embodiment is a method of converting biogas to liquid oxygenates comprising methanol and acetic acid by plasma-treated metal-based mesoporous and microporous materials under plasma catalytic system, where the liquid products can be tailored by SBA-15 and ZSM-5. When the packed catalyst is plasma-treated ZSM-5, acetic acid is the dominating liquid product with a high relative selectivity. When Cu is loaded on SBA-15, the dominating liquid product is reversed from acetic acid to methanol.

### BRIEF DESCRIPTION OF THE DRAWINGS

FIG. 1A shows a bar chart indicating the effect of different catalysts on the relative selectivity for main liquid products during plasma catalytic conversion of CH₄ and CO₂.

FIG. 1B shows a plot of the relative selectivity towards methanol and acetic acid as a function of Cu loading on alumina for the main liquid products; and stars indicate their relative selectivity for liquid products in plasma only mode.

FIG. 1C shows images using FESEM and EDS for plasma reduced Cu/Al₂O₃ catalyst.

FIG. 2 shows a plot of the relative selectivity for main liquid products vs the discharge power for biogas conversion in plasma only mode.

FIG. 3 is a plot of the relative selectivity for main liquid products vs CO₂:CH₄ ratio in plasma only mode.

FIG. 4A is a plot of the relative selectivity for main liquid products vs GHSV in plasma only mode.

FIG. 4B is a plot of the relative selectivity for main liquid products vs GHSV in plasma+Cu/Al₂O₃ packing mode.

FIG. 5A shows a bar chart of the conversion rate of CO₂ and CH₄ in biogas without water addition and with water addition.

FIG. 5B shows a bar chart of the relative selectivity for methanol, ethanol and acetic acid with a biogas without water and with water addition.

FIG. 6A shows a bar chart of the conversion rate of CO₂ and CH₄ in plasma biogas conversion using different mesoporous and microporous catalysts.

FIG. 6B shows a bar chart of the relative selectivity for main liquid products in plasma biogas conversion using different mesoporous and microporous catalysts.

### DETAILED DISCLOSURE OF THE INVENTION

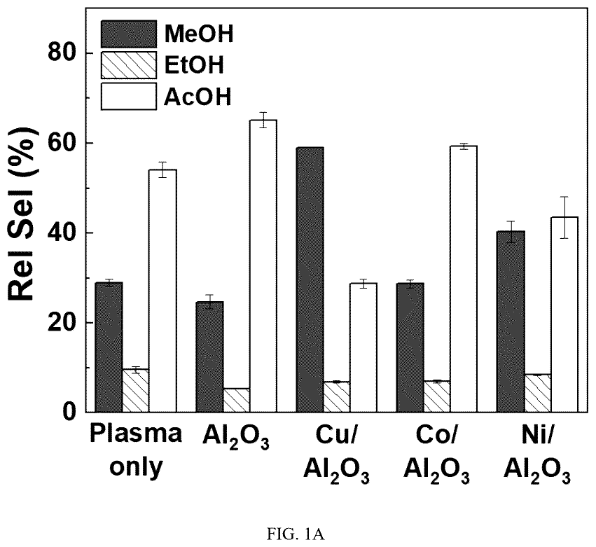

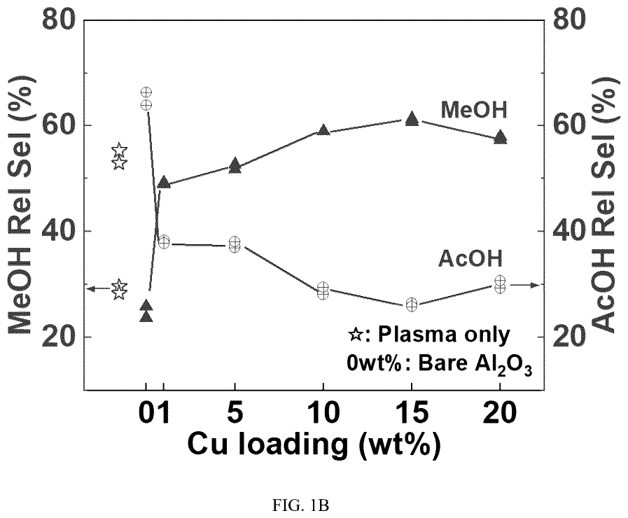

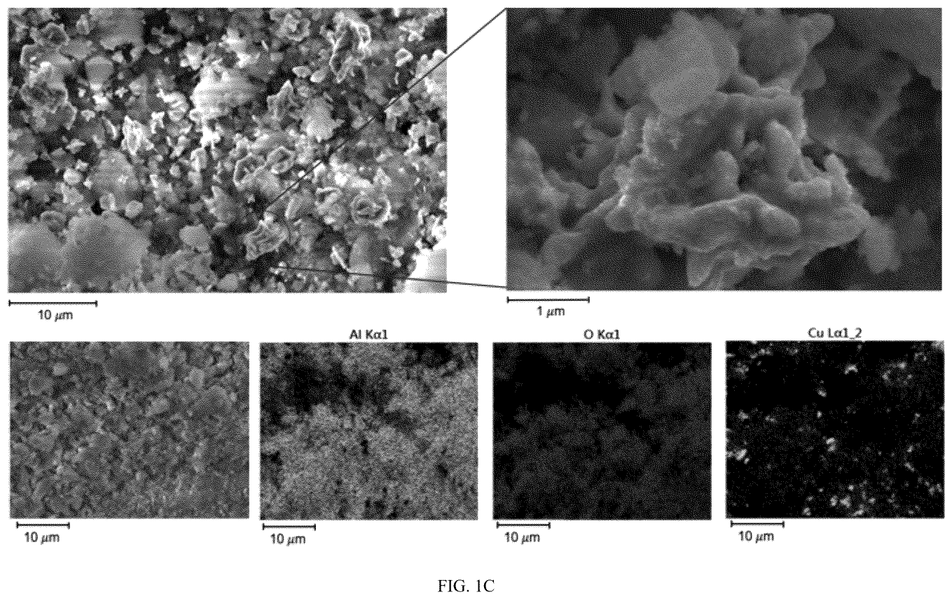

Embodiments are to an integrated plasma catalysis system for biogas conversion biased for production of acetic acid and methanol in varied proportions. Biogas from various solid waste sources containing two greenhouse gases, methane and carbon dioxide, can be converted efficiently to useful chemicals. In embodiments, plasma-reduced metallic catalysts including Cu/γ-Al₂O₃, Co/γ-Al₂O₃, and/or Ni/γ-Al₂O₃ can form the liquid products acetic acid, methanol, and ethanol, with trace amounts of propanol, butanol, formaldehyde and acetone under plasma. In embodiments the Cu/γ-Al₂O₃ catalyst leads to a methanol-dominated liquid product. As shown in FIG. 1A, metal-loaded γ-Al₂O₃-based catalysts result in different distributions of liquid oxygenates from bare γ-Al₂O₃ support. Acetic acid dominates liquid products for most metal-based catalyst packings and with bare γ-Al₂O₃ packing, which exhibits the highest relative selectivity for acetic acid. In contrast, a packing of Cu/Al₂O₃ produces methanol as a dominating liquid product. Loading of copper results in a significantly higher relative selectivity for methanol than plasma only and the other packings. The effect of Cu/Al₂O₃ with different Cu loadings at a discharge power of 20 W, as shown in FIG. 1B, indicates that Cu loading has a remarkable influence on liquid product distribution. Even a 0.1 wt % Cu loading enhances the selectivity for methanol apparently, demonstrating the unique role of Cu in promoting methanol synthesis. Increasing Cu loading from 0.1 wt % to 15 wt % further enhances the selectivity for methanol over acetic acid. Methanol selectivity drops slightly when the loading rises further to 20 wt %. Moreover, the morphology of the catalyst surface can affect the distribution of liquid products. A unique flower-like morphology of copper species on Cu/Al₂O₃ due to plasma reduction, as shown in FIG. 1C, appears to favor methanol production, whereas thermally-reduced Cu/Al₂O₃ does not exhibit this flower-like morphology. Herein, Cu/Al₂O₃ indicates the plasma reduced flower-like catalyst unless otherwise indicated.

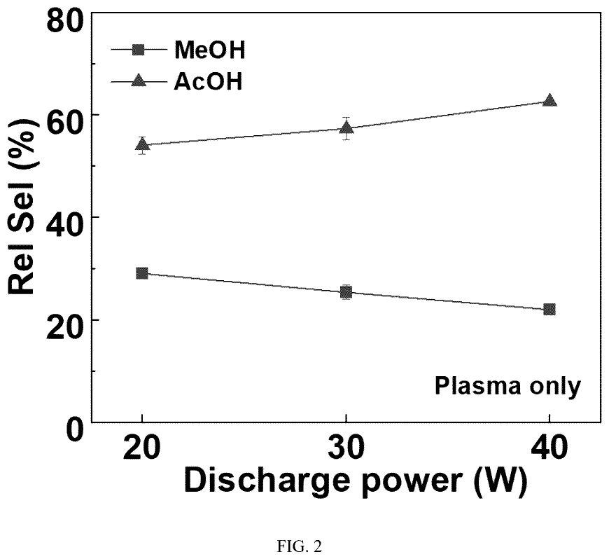

In situ optical emission spectroscopy of CO₂—CH₄ plasma proved the presence of various reactive species in the discharge zone, such as CO₂⁺, H*, CO*, and O*, where * indicates a radical. The combination of the reactive species on the catalyst surface and in plasma phase leads to the formation of oxygenates and other products. Consequently, Cu appears to have a moderate binding strength towards the key intermediates for methanol formation, such as HCOO*, CH₃* and CH₃O*, leading to direct combination of radicals on the Cu surface to form methanol as the primary product. In addition to the catalyst, plasma itself is another key factor influencing the selectivity for liquid oxygenates. Discharge power affects the liquid products composition to different extents with and without a catalyst packing. As shown in FIG. 2, discharge power slightly affects the relative selectivity for liquid products in plasma with no catalyst. In contrast, the presence of Cu significantly influences the distribution of liquid products. Packing Cu/Al₂O₃ in the discharge zone dramatically changes the dominating liquid product, where the dominant product reverses from methanol to acetic acid as the discharge power increases from 20 W to 40 W. Discharge power becomes a critical factor to the distribution of liquid products with a catalyst packing in the discharge zone. With the increase of discharge power, the electric field strengthens such that the adsorption of intermediates and the desorption of products on the catalyst surface is influenced, affecting the catalytic effect of metal-based catalysts.

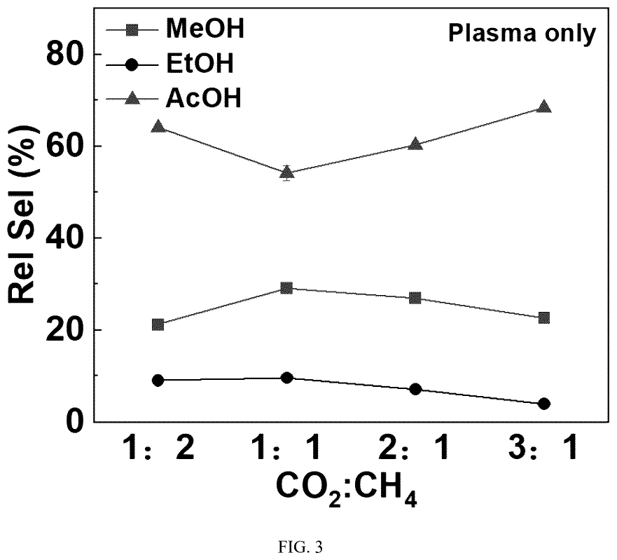

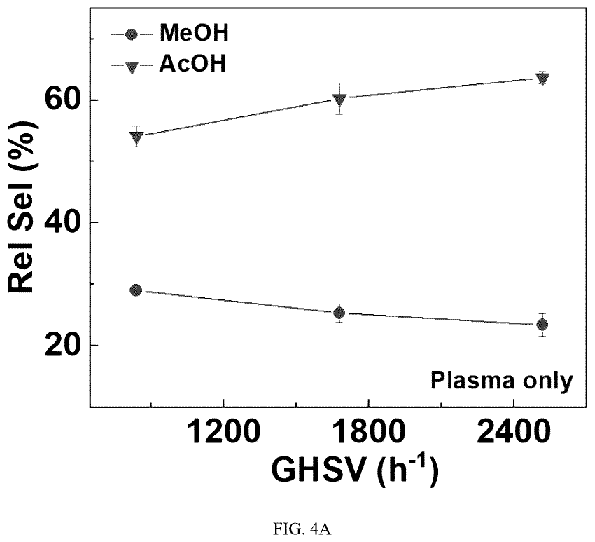

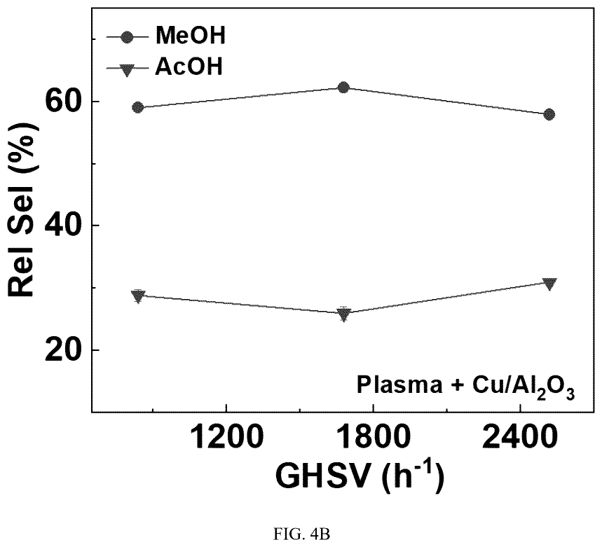

As shown in FIG. 3, the CO₂:CH₄ ratio for best methanol selectivity is 1:1 in plasma only case. However, with Cu/Al₂O₃ packing in the discharge zone, the optimum CO₂:CH₄ ratio becomes 2:1 for best methanol selectivity at a much higher level, indicating the strong promotion of Cu/Al₂O₃ catalyst for methanol. Additionally, the gas-hourly space velocity (GHSV) influences the liquid product distribution as indicated in FIG. 4A and FG. 4B. In plasma only case, the relative selectivity for methanol drops with the increase of GHSV. Without a catalyst, increasing GHSV increases acetic acid selectivity but decreases methanol selectivity. However, packing Cu/Al₂O₃ catalyst keeps the relative selectivity for methanol at a much higher level with the increase of GHSV, indicating the significant role Cu catalyst plays in the secondary reactions of products.

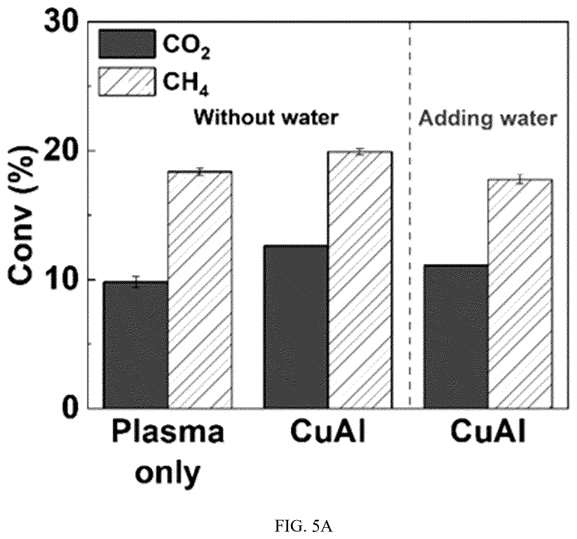

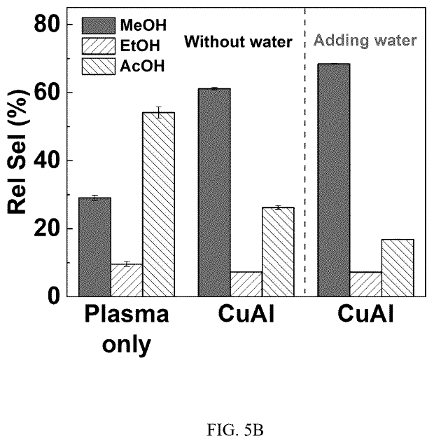

A certain amount of water addition turns out to be another way to tune the liquid product selectivity. Adding a little amount of water (in the range of 1% to 5%) into the feed gas does not lead to a significant decrease in reactant conversion, as shown in FIG. 5A, but significantly facilitates the production of methanol in both plasma only case and Cu/Al₂O₃ packing case, as shown in FIG. 5B.

Embodiments of the invention are directed to tailoring the distribution of liquid products in plasma catalytic conversion of biogas by: tuning the discharge power; the type and nature of the metal-based catalyst; and control of the process parameters. The type of metals and the nature of catalyst surface are critical. Plasma-reduced Cu/Al₂O₃ promotes methanol production. The copper catalyst facilitates direct combination of radicals to form methanol by binding with CH₃* and CH₃O* intermediates, promotes CO₂ hydrogenation to methanol, and enables conversion of produced acetic acid to methanol under plasma. From the perspective of plasma, discharge power can modify the proportions of liquid products and even reverse the dominating liquid product between methanol and acetic acid in the presence of the Cu-based catalysts. Discharge power may influence the catalytic effect by its electric field. Process parameters useful for tuning the process include the CO₂:CH₄ ratio and gas hourly space velocity (GHSV), which influence the selectivity for liquid products, apparently by affecting the coverage and residence time of radicals or products. Water vapor addition can be a good way to facilitate methanol production under plasma. Among these three factors, the effect of process parameters paled by comparison with discharge power and metal-based catalysts, and discharge power was identified as the most critical factor dictating the selectivity for liquid products.

The Cu/γ-Al₂O₃ catalyst is prepared by incipient impregnation followed by H₂ plasma treatment using a H₂ flowrate of 10 to 100 mL/min and with a discharge power of 10 to 40 W where the discharge gap is 1 to 4 mm and the discharge length is 1 to 10 cm during a plasma treatment period of one to four hours. The copper loading can vary from 1 to 25 wt %.

The catalyst promoting methanol production is plasma-treated Cu/γ-Al₂O₃, wherein the loading amounts of copper leading to the highest methanol selectivity are between 5 wt % and 20 wt % copper. The catalysts promoting acetic acid production are plasma-treated bare γ-Al₂O₃ and Co/γ-Al₂O₃ with a cobalt loading of 5 to 20 wt %. The plasma reactor can be a dielectric barrier discharge (DBD), wherein the discharge gap is between 1 to 4 mm, discharge length is between 1 to 10 cm, and discharge power of 5 to 45 W to promote methanol production. A discharge power of 15 to 55 W can be used to promote acetic acid production. A frequency of 7 kHz to 10 kHz is effective. An optimal CH₄:CO₂ volume ratio for methanol production is 0.2 to 3, and the optimal CH₄:CO₂ volume ratio for acetic acid production is 0.1 to 5 with the appropriate plasma and catalyst. The gas hourly space velocity (GHSV) can be 700 to 2700 h⁻¹. Water addition to the biogas can be 1 to 15 vol %, which is useful for methanol production. Temperature can range from 0 to 240° C. for the production of methanol and 10 to 250° C. for the production of acetic acid.

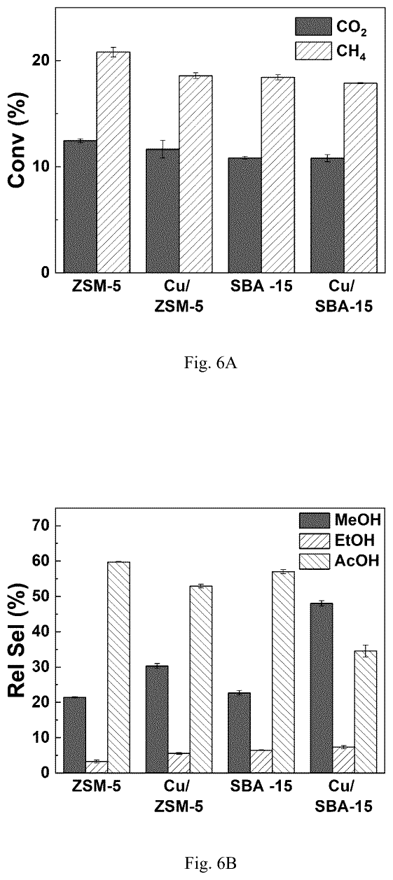

Another group of catalyst for biogas conversion are mesoporous and microporous materials including SBA-15 and ZSM-5 to tailor the liquid products. The conversion rates of CO₂ and CH₄ in biogas on SBA-15 and ZSM-5 catalysts are similar to gamma alumina-based catalysts, as shown in FIG. 6A. When the packed catalyst is plasma-treated ZSM-5 or SBA-15, acetic acid is the dominating liquid product with a high relative selectivity, as shown in FIG. 6B. When Cu is loaded on SBA-15, the dominating liquid product is reversed from acetic acid to methanol.

### EXEMPLARY EMBODIMENTS

Embodiment 1. An integrated plasma catalysis system for biogas conversion, comprising a plasma-treated mesoporous and microporous catalyst including gamma alumina supported catalyst, SBA-15 or ZSM-5, and a plasma source wherein the biogas comprising methane and carbon dioxide is converted to liquid oxygenates mainly comprising methanol and acetic acid.

Embodiment 2. The integrated plasma catalysis system for biogas conversion according to embodiment 1, wherein mesoporous and microporous catalysts including gamma alumina supported catalyst, SBA-15 and ZSM-5 are optionally impregnated with a metal and treated by hydrogen plasma.

Embodiment 3. The integrated plasma catalysis system for biogas conversion according to embodiment 2, wherein the metal comprises copper, cobalt, nickel, or any combination thereof.

Embodiment 4. The integrated plasma catalysis system for biogas conversion according to embodiment 3, wherein the metal resides in a flower-like morphology.

Embodiment 5. The integrated plasma catalysis system for biogas conversion according to embodiment 1, wherein the plasma source is a dielectric barrier discharge (DBD) plasma reactor.

Embodiment 6. A method for preparing the plasma-treated mesoporous and microporous supported catalyst according to embodiment 1, comprising:
providing a catalyst support;
optionally impregnating the catalyst support with a metal source; and treating the metal-loaded catalyst with a hydrogen plasma.

Embodiment 7. The method according to embodiment 6, wherein the metal source comprises a copper source, a cobalt source, or a nickel source.

Embodiment 8. The method according to embodiment 6, wherein the hydrogen plasma is supported by a hydrogen flow rate in a range of from 10 to 100 mL/min.

Embodiment 9. The method according to embodiment 6, wherein the hydrogen plasma employs a discharge power in a range of from 10 to 40 W.

Embodiment 10. The method according to embodiment 6, wherein hydrogen plasma employs a discharge gap in a range of from 1 to 4 mm and the discharge length is in a range of from 2 to 7 cm.

Embodiment 11. The method according to embodiment 6, wherein plasma treatment is for a period of 1 to 4 hours.

Embodiment 12. A method of converting biogas to liquid oxygenates such as methanol and acetic acid, comprising:
providing an integrated plasma catalysis system according to embodiment 1;
providing a biogas comprising methane and carbon dioxide to the plasma-treated mesoporous or microporous catalyst of the integrated plasma catalysis system;
treating the biogas with a plasma generated by the plasma source of the integrated plasma catalysis system; and
tailoring liquid oxygenates between methanol and acetic acid.

Embodiment 13. The method according to embodiment 12, wherein the plasma-treated mesoporous or microporous catalyst comprises plasma treated γ-Al₂O₃, Co/γ-Al₂O₃, Ni/γ-Al₂O₃, Cu/γ-Al₂O₃, SBA-15, Cu/SBA-15, ZSM-5, Cu/ZSM-5 or any combination thereof.

Embodiment 14. The method according to embodiment 13, wherein the plasma-treated gamma alumina supported catalyst comprises plasma treated γ-Al₂O₃, plasma treated Co/γ-Al₂O₃, or plasma treated Ni/γ-Al₂O₃ wherein the Co or Ni are at a 5 to 20 wt % loading wherein the liquid oxygenates comprises more acetic acid than methanol.

Embodiment 15. The method according to embodiment 13, wherein the plasma-treated gamma alumina supported catalyst comprises plasma treated Cu/γ-Al₂O₃ wherein the Cu is at a 1 to 25 wt % loading, wherein the liquid oxygenates comprises more methanol than acetic acid.

Embodiment 16. The method according to embodiment 12, wherein the plasma source comprises a dielectric barrier discharge (DBD) reactor.

Embodiment 17. The method according to embodiment 16, wherein, the dielectric barrier discharge (DBD) reactor employs a discharge gap in a range of from 1 to 4 mm and a discharge length in a range of from 2 to 7 cm.

Embodiment 18. The method according to embodiment 16, wherein, the dielectric barrier discharge (DBD) reactor employs a discharge power in a range of from 5 to 45 W for a methanol rich liquid oxygenates or a discharge power in a range of from 15 to 55 W for an acetic acid rich liquid oxygenates.

Embodiment 19. The method according to embodiment 16, wherein a frequency of the dielectric barrier discharge (DBD) reactor is in a range of from 7 kHz to 10 KHz.

Embodiment 20. The method according to embodiment 12, wherein the biogas comprises a CH₄:CO₂ ratio of 0.2 to 3 for a methanol rich liquid oxygenates or a CH₄:CO₂ ratio of 0.1 to 5 for an acetic acid rich liquid oxygenates.

Embodiment 21. The method according to embodiment 12, wherein the gas-hourly space velocity (GHSV) is in a range of from 700 to 2700 h⁻¹.

Embodiment 22. The method according to embodiment 12, wherein the temperature is in a range of from 0 to 240° C. for a methanol rich liquid oxygenates and the temperature is in a range of from 10 to 250° C. for an acetic acid rich liquid oxygenates.

Embodiment 23. The method according to embodiment 12, further comprising feeding water at 1% to 15% to the biogas.

Embodiment 24. The method according to embodiment 13, wherein the plasma-treated ordered mesoporous catalyst comprises plasma treated SBA-15, and plasma treated Cu/SBA-15 wherein the Cu is at a 0.1 to 20 wt % loading.

Embodiment 25. The method according to embodiment 13, wherein the plasma-treated ordered microporous catalyst comprises plasma treated ZSM-5, and plasma treated Cu/ZSM-5 wherein the Cu is at a 0.1 to 20 wt % loading.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

I. E. Agency, Outlook for biogas and biomethane: Prospects for organic growth, OECD Publishing, 2020.
D. S. Grégoire, N. A. George, L. A. Hug, Nature Communications 2023, 14, 7402.
C. Song, in CO2 Conversion and Utilization, Vol. 809, American Chemical Society, 2002, pp. 2-30.
T. J. Crowley, R. A. Berner, Science 2001, 292, 870-872.
C. Song, Catalysis today 2006, 115, 2-32.
Y. Wang, Y. Chen, J. Harding, H. He, A. Bogaerts, X. Tu, Chemical Engineering Journal 2022, 450, 137860.
Y. Zeng, L. Wang, C. Wu, J. Wang, B. Shen, X. Tu, Applied Catalysis B: Environmental 2018, 224, 469-478.
C. Palmer, D. C. Upham, S. Smart, M. J. Gordon, H. Metiu, E. W. McFarland, Nature Catalysis 2020, 3, 83-89.
E. K. Gibson, C. E. Stere, B. Curran-McAteer, W. Jones, G. Cibin, D. Gianolio, A. Goguet, P. P. Wells, C. R. A. Catlow, P. Collier, Angewandte Chemie International Edition 2017, 56, 9351-9355.
C. E. Stere, J. A. Anderson, S. Chansai, J. J. Delgado, A. Goguet, W. G. Graham, C. Hardacre, S. R. Taylor, X. Tu, Z. Wang, Angewandte Chemie International Edition 2017, 56, 5579-5583.
L. Wang, Y. Yi, C. Wu, H. Guo, X. Tu, Angewandte Chemie International Edition 2017, 56, 13679-13683.
J. Wang, M. S. AlQahtani, X. Wang, S. D. Knecht, S. G. Bilén, C. Song, W. Chu, Green Chemistry 2021, 23, 1642-1647.
R. Snoeckx, A. Bogaerts, Chemical Society Reviews 2017, 46, 585-5863.
U. Kogelschatz, B. Eliasson, W. Egli, Le Journal de Physique IV 1997, 7, C4-47-C44-66.
J.-J. Zou, Y.-p. Zhang, C.-J. Liu, Y. Li, B. Eliasson, Plasma Chemistry and Plasma Processing 2003, 23, 69-82.
R. Pang, K. Teramura, M. Morishita, H. Asakura, S. Hosokawa, T. Tanaka, Communications Chemistry 2020, 3, 137.
J. Wang, X. Wang, M. S. AlQahtani, S. D. Knecht, S. G. Bilen, W. Chu, C. Song, Chemical Engineering Journal 2023, 451, 138661.
Y. Zeng, G. Chen, Q. Bai, L. Wang, R. Wu, X. Tu, International Journal of Hydrogen Energy 2023, 48, 6192-6203.
D. Mei, X. Shen, S. Liu, R. Zhou, X. Yuan, Z. Rao, Y. Sun, Z. Fang, X. Du, Y. Zhou, Chemical Engineering Journal 2023, 462, 142044.
J. Li, L. Dou, Y. Liu, Y. Gao, X. Hu, F. Yu, J. Li, S. Zhang, T. Shao, Fuel Processing Technology 2023, 242, 107648.
D. Li, V. Rohani, F. Fabry, A. P. Ramaswamy, M. Sennour, L. Fulcheri, Applied Catalysis B: Environmental 2020, 261, 118228.
B. Loenders, R. Michiels, A. Bogaerts, Journal of Energy Chemistry 2023.
H. Zhao, R. Yu, S. Ma, K. Xu, Y. Chen, K. Jiang, Y. Fang, C. Zhu, X. Liu, Y. Tang, Nature Catalysis 2022, 5, 818-831.
M. Y. Abdul Halim, W. L. Tan, N. H. H. Abu Bakar, M. Abu Bakar, Materials 2014, 7, 7737-7751.
F. Studt, I. Sharafutdinov, F. Abild-Pedersen, C. F. Elkjær, J. S. Hummelshøj, S. Dahl, I. Chorkendorff, J. K. Nørskov, Nature chemistry 2014, 6, 320-324.
C. F. Nwaokorie, M. M. Montemore, The Journal of Physical Chemistry C 2022, 126, 3993-3999.
J. Andersen, J. Christensen, M. Østberg, A. Bogaerts, A. Jensen, Chemical engineering journal 2020, 397, 125519.
X. Jiang, N. Koizumi, X. Guo, C. Song, Applied Catalysis B: Environmental 2015, 170, 173-185.
Y.-R. Zhang, E. C. Neyts, A. Bogaerts, The Journal of Physical Chemistry C 2016, 120, 25923-25934.
Z. Cui, S. Meng, Y. Yi, A. Jafarzadeh, S. Li, E. C. Neyts, Y. Hao, L. Li, X. Zhang, X. Wang, ACS Catalysis 2022, 12, 1326-1337.
M. S. Usman, N. A. Ibrahim, K. Shameli, N. Zainuddin, W. M. Z. W. Yunus, Molecules 2012, 17, 14928-14936.

We claim:

1. An integrated plasma catalysis system for biogas conversion, comprising a plasma-treated mesoporous or microporous catalyst and a plasma source wherein a biogas comprising methane and carbon dioxide is converted to liquid oxygenates comprising mainly methanol and acetic acid.

2. The integrated plasma catalysis system for biogas conversion according to claim 1, wherein the mesoporous or microporous catalysts is selected from gamma alumina, SBA-15 and ZSM-5 supported catalyst, optionally impregnated with a metal and treated by hydrogen plasma.

3. The integrated plasma catalysis system for biogas conversion according to claim 2, wherein the metal comprises copper, cobalt, nickel, or any combination thereof.

4. The integrated plasma catalysis system for biogas conversion according to claim 3, wherein the metal resides in a flower-like morphology.

5. The integrated plasma catalysis system for biogas conversion according to claim 1, wherein the plasma source is a dielectric barrier discharge (DBD) plasma reactor.

6. A method for preparing the plasma-treated mesoporous or microporous catalyst according to claim 1, comprising:
providing a gamma alumina SBA-15, or ZSM-5 support;
optionally impregnating the mesoporous or microporous support with a metal source; and
treating the mesoporous or microporous catalyst with a hydrogen plasma.

7. The method according to claim 6, wherein the metal source comprises a copper source, a cobalt source, or a nickel source.

8. The method according to claim 6, wherein the hydrogen plasma is supported by a hydrogen flow rate in a range of from 10 to 100 mL/min.

9. The method according to claim 6, wherein the hydrogen plasma employs a discharge power in a range of from 10 to 40 W.

10. The method according to claim 6, wherein hydrogen plasma employs a discharge gap in a range of from 1 to 4 mm and the discharge length is in a range of from 1 to 10 cm.

11. The method according to claim 6, wherein treating is for a period of 1 to 4 hours.

12. A method of converting a biogas to liquid oxygenates comprising methanol and acetic acid, comprising:
providing an integrated plasma catalysis system according to claim 1;
providing a biogas comprising methane and carbon dioxide to the plasma-treated mesoporous or microporous catalyst of the integrated plasma catalysis system;
treating the biogas with a plasma generated by the plasma source of the integrated plasma catalysis system; and
tailoring the ratio between methanol and acetic acid in liquid oxygenates.

13. The method according to claim 12, wherein the plasma-treated mesoporous or microporous comprises plasma treated γ-Al₂O₃, Co/γ-Al₂O₃, Ni/γ-Al₂O₃, Cu/γ-Al₂O₃, SBA-15, Cu/SBA-15, ZSM-5, Cu/ZSM-5 or any combination thereof.

14. The method according to claim 13, wherein the plasma-treated mesoporous or microporous catalyst comprises plasma treated γ-Al₂O₃, plasma treated Co/γ-Al₂O₃, or plasma treated Ni/γ-Al₂O₃ wherein the Co or Ni are at a 5 to 20 wt % loading wherein the liquid oxygenates comprises more acetic acid than methanol.

15. The method according to claim 13, wherein the plasma-treated mesoporous or microporous catalyst comprises plasma treated Cu/γ-Al₂O₃ wherein the Cu is at a 0.1 to 25 wt % loading, wherein the liquid oxygenates comprises more methanol than acetic acid.

16. The method according to claim 12, wherein the plasma source comprises a dielectric barrier discharge (DBD) reactor.

17. The method according to claim 16, wherein, the dielectric barrier discharge (DBD) reactor employs a discharge gap in a range of from 1 to 4 mm and a discharge length in a range of from 1 to 10 cm.

18. The method according to claim 16, wherein, the dielectric barrier discharge (DBD) reactor employs a discharge power in a range of from 5 to 45 W for a methanol rich liquid oxygenates or a discharge power in a range of from 15 to 55 W for an acetic acid rich liquid oxygenates.

19. The method according to claim 16, wherein a frequency of the dielectric barrier discharge (DBD) reactor is in a range of from 7 kHz to 10 kHz.

20. The method according to claim 12, wherein the biogas comprises a CH₄:CO₂ ratio of 0.2 to 3 for a methanol rich liquid oxygenates or a CH₄:CO₂ ratio of 0.1 to 5 for an acetic acid rich liquid oxygenates.

21. The method according to claim 12, wherein the gas flow (GHSV) is in a range of from 700 to 2700 h⁻¹.

22. The method according to claim 12, wherein a temperature is in a range of from 0 to 240° C. for a methanol rich liquid oxygenates and the temperature is in a range of from 10 to 250° C. for an acetic acid rich liquid oxygenates.

23. The method according to claim 12, further comprising feeding water vapor at 1% to 15% of the total volume of the biogas, to the biogas.

24. The method according to claim 12, wherein the plasma-treated ordered mesoporous catalyst is selected from plasma treated SBA-15, and plasma treated Cu/SBA-15, wherein the Cu is at a 0.1 to 20 wt % loading.

25. The method according to claim 13, wherein the plasma-treated ordered microporous catalyst is selected from plasma treated ZSM-5, and plasma treated Cu/ZSM-5, wherein the Cu is at a 0.1 to 20 wt % loading.
